(19) United States
(12) Patent Application Publication
(10) Pub. No.: US 2003/0038920 A1
(43) Pub. Date: Feb. 27, 2003

(54) Apparatus and methods for vision correction using refractive index effects

(76) Inventor: J. T. Lin, Oviedo, FL (US)

Correspondence Address: J. T. LIN, 4532 Old Carriage Trail, Oviedo, FL, 32765, US

(21) Appl. No.: 09/932,959

(22) Filed: Aug. 21, 2001

(51) Int. Cl. A61B 3/10

(52) U.S. Cl. 351/212

(57) ABSTRACT

A method and apparatus for performing vision correction by selecting means of changing the refractive index includes medical, mechanical, optical or chemical method. Detail theoretical calculation with clinical predictions are presented for quantitative index changes required to correct myopia, hyperopia and presbyopia. The key parameters of the radius and thickness of the cornea, lens and indices of the lens, cornea and humor chambers and the effect due aging are proposed.

### BACKGROUND OF THE INVENTION

1. Field of the Invention

The present invention relates to methods and apparatus for vision correction by modifying the refractive index of the eye to change the image positions and for the treatment of myopia, hyperopia, astigmatism and presbyopia.

2. Prior Art

Corneal reshaping including procedures of photorefractive keratectomy (PRK) or laser assisted in situ keratomileusis, or laser intrastroma keratomileusis (LASIK) have been proposed by many or the prior arts such as U.S. Pat. Nos. 4,718,418, 4,840,175, 4,856,513, 4,988,348, 5,019,074, 5,520,679, 5,928,129. All of these prior arts are using an ablation laser means to reshape the corneal central portion curvature in order to correct patient's visions. The above-described prior arts using lasers to reshape the corneal surface curvature, however, are limited to the corrections of myopia, hyperopia and astigmatism.

Refractive surgery using a scanning device and lasers in the mid-infrared (mid-IR) wavelength was first proposed by the present inventor in U.S. Pat. No. 5,144,630 and 5,520,679 and later proposed by Telfair et. al., in U.S. Pat. No. 5,782,822. Corneal reshaping may also be performed by laser thermal coagulation currently conducted by a Ho:YAG laser (at about 2 microns in wavelength) proposed by Sand in U.S. Pat. 5,484,432. Ruiz in U.S. Pat. No. 5,533,997 proposed the use of laser ablation of cornea surface to correct presbyopic patients. This prior art, however, must generate multifocal (or bifocal) surface on the central portion of the cornea in order to achieve the desired presbyopia correction.

All the above-described prior arts are using methods to change the cornea surface curvature either by tissue ablation (such as in UV laser) or by thermal shrinkage (such as in Ho:YAG laser) and all are using lasers onto the central potion of the cornea.

Correction of presbyopia via the expanding of the sclera by mechanical devices was recently proposed by Schachar in U.S. Pat. Nos. 5,489,299 and 5,354,331. These prior arts all require the implant of external band or using laser heating to affect the position of the insertion band and have the drawbacks of complexity, time consuming, costly and potential for side effects. To treat presbyopia, Schachar's other U.S. Pat. Nos. 5,529,076 and 5,722,952, proposed the use of heat or radiation on the corneal epithelium to arrest the growth of the crystalline lens by laser coagulation effects. The alternative methods for presbyopia correction by changing the intrinsic properties of the sclera and cililary tissue to increase the lens accommodation without changing the cornea curvature were recently proposed by the present inventor in U.S. Pat. No. 6,258,082 and 6,263,879.

None of the prior arts above-described was dealing with the refractive index inside the eye. Accordingly, there is a strong need to correct patient's visions without reshaping the cornea or conducting surgery on the sclera tissue. A totally non-invasive method is proposed in the present patent by modifying the refractive index of the eye, where an effective focal length of the eye is defined to change the image position. The position of the retinal image varies from patient to patient and may also change by aging. Human's vision condition is also changed by the physical and chemical properties of the cornea, lens and the anterior and posterior chambers of the eye, the aqueous humor and the vitreous chamber.

It is yet another objective of the present patent is to provide a quantitative theory as the guidance for the correction of myopia, hyperopic, astigmatism and presbyopia.

It is yet another objective of the present patent is to provide the refractive index method as an enhancement for the post-operative patients conducted by the other methods such as LASIK and LTK.

### SUMMARY OF THE INVENTION

Human's vision condition is changed by the physical and chemical properties of the cornea, lens and the anterior and posterior chambers of the eye, the aqueous humor and the vitreous chamber.

The present patent is to provide a quantitative theory as the guidance forthe correction of myopia, hyperopic, astigmatism and presbyopia. These theoretical guidance includes the equations provided for the patients diopter vs the image position, vs. the radius, thickness, separation and indices changes of the lens, cornea and humor chambers. We predict some key clinical issues for the correction of myopia, hyperopia and presbyopia based on the theory developed in the present patent. Change of the index may be provided by the preferred embodiments including medical, mechanical, optical and chemical methods.

It is yet another preferred embodiments of the present patent is to provide the refractive index method as an enhancement for the post-operative patients conducted by the other methods such as LASIK and LTK.

Further preferred embodiments of the present invention will become apparent from the description of the invention which follows.

### BRIEF DESCRIPTION OF THE DRAWINGS

FIG. 1 is a block diagram of an eye model showing light propagating through various portion of the eye, the cornea, the anterior chamber, the lens and the posterior chamber. The image position for uncorrected patient id shifted to the retinal position.

### DETAILED DESCRIPTION OF THE INVENTION AND THE PREFERRED EMBODIMENTS

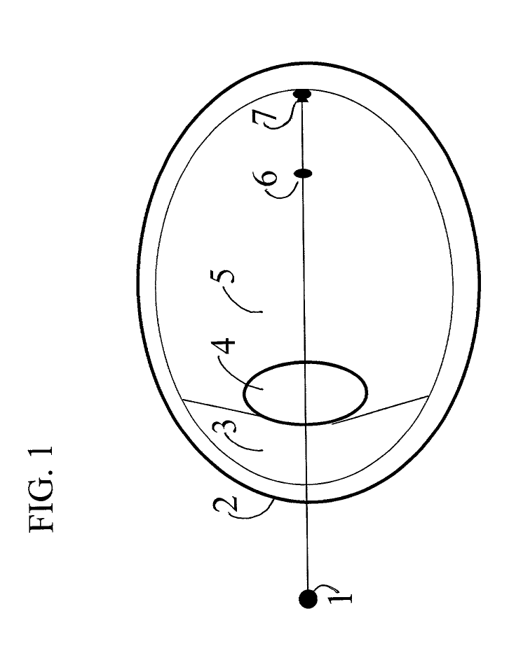

Referring to FIG. 1, an eye model showing light propagating starting from an object position 1 through various portion of the eye, the cornea 2, the anterior chamber 3, the lens 4 and the vitreous chamber 5. The image's position was indicated by 6 before the index change and 7 after the refractive index changed, where location 7 is the retinal image position. We note that for myopia (hyperopia) patient, the image position 6 is shorter (longer) than the retinal position 7. To correct patient's vision, the index change shall shift the image position from 6 to 7.

Still referring to FIG. 1, light propagating starting from a position 1 through the cornea anterior surface with a radius of r1 which causes the incoming light to be focused. The posterior radius of the cornea r2 will cause the light to diverge due to its concave nature of the cornea. The incoming light is then pass the aqueous humor 3 and the lens 4 before going to the vitreous chamber 5. The lens 4 is a converging focusing lens which focuses the light onto the image position 6. This uncorrected image position 6 may be inside or outside the retinal image position 7 depending on the patient's uncorrected vision is myopic or hyperopic.

We shall now present an effective focal length (F) of an eye which defines the image position at a given set of condition of the cornea, lens and chambers. We first define the refractive index of each portion of the eye by nc (for cornea), n1 (for anterior chamber), n (for the lens) and n2 (for the vitreous chamber). The radius of the cornea (and lens) by r1 (and R1) for the anterior surface and r2 (and R2) for the posterior surface. By the light propagation theory (referred to Chapter 4 in “Principle of Optics”, by Max Born and Emil Wolf, Fourth Edition, 1970) and for thick lens, assuming the thickness of the cornea and lens are tc and t, and separated by a distance S, we are able to derive the following

n2/F=n1/f1+n2/f2−n2 S/(f1 f2), (1)

where f1 and f2 are the effective focal length of the cornea and the lens, respectively and are further given by

n1//f1=(nc−1)/r1−(nc−n1)/r2+(nc−1) (nc−n1)tc/(nc r1 r2), (2.a)

n1//f1=(n−n1 )/R1−(n−n2)/R2+(n−n1) (n−n2)t/(n R1 R2), (2.b)

noting that all radius are positive, except r2 which is negative for the posterior surface of the lens. Moreover, the positions of the object (a) and the image (b) are related to the effective focal length (F) of the eye defined by combined focal length of the cornea (f1) and the lens (f2) as follows:

1/a+n1/b=n1/F. (3)

Now we are looking for the effects of the physical parameters of an eye and the position of the image which for a piano patient shall be about 24.38 mm (defined as B), that is the position of the retinal, or the length of the eye ball. We note that b<B, fro myopic patient and b>B for hyperopia or presbyopia. The diopter (D) defining the degree of myopia (D is negative) and hyperopia (D is positive) is given by

D=−1000(1/a1−1/a), (4.a)

which becomes, by substituting Eq. (3),

D=1000[n1 (1/b1−1/b)+n2 (1/F- 1/F1)]. (4.b)

Where b1 and F1 are the corresponding image position and effective focal length for object position al, all are in the unit of mm. Therefore we may define the image difference, or the image shifting db=(b1−b), and when using b1=B=retinal position=24.38 mm, this shifting db provides us the amount of image potion deviates from the piano position of an eye. For a given effective focal length F, this image shift (db) is related to the diopter (D) by

db=−0.445 D [1−0.018 D], (5)

where we have used an approximate expression of the db vs. D which is accurate within 1% error comparing with the exact analytic equation. We note that for a=1.0 meter or 1000 mm, D=−1.0 and −2.0 for the object at a1=0.5 meter and 0.25 meter, respectively.

Now we will provide some analytic equations which relate D to the key parameters of the eye including the changes of refractive indices (nc, n1, n), the radius (r1, r2) of the cornea and lens (R1, R2), the separation between the lens and the cornea, and their thickness (tc, t). For easier analysis, we further define the percentage ratio of the indices changes given by

Xc=nc′/nc , X=n′/n , X1=n1′/n1, (6)

which provide the index change ratio of the new index with respect to the original. Using the expressions of Eq. (6) and substituting Eqs. (1) and (2) into Eq. (4), we obtain an overall expression for the diopter required for a patient D as follows:

D=D1+D2+D3+D4+D5, (7)

where each components of D is governed by various parameters changes of the eye as follows

D1=377 (dr1) [1−S/f2+0.0046 tc] (8.a)

D2=83 (dR1+dR2) (8.b)

D3=−40 (Xc−1) (8.c)

D4=+365 (X−1) (8.d)

D5=−142 (X1−1). (8.e)

Where we have used the known parameters in deriving above analytic equations (all in the units of mm): R1=10.2, R2=6.0, r1=7.8, r2=6.5, nc=1.377, n1=n2=1.337, n=1.42. The derivatives are defined by the difference of reverse of each parameters as: dr1=1/r1′−1/r1, dR1=1/R1′−1/R1 etc.

Based on above equations, we are now able to address some clinically important issues for correcting a human vision as follows:

(a) The first term, D1, is actually provides the diopter correction needed for the procedure of PRK or LASIK which changes the cornea anterior curvature by a laser ablation of the cornea, except that we have further included the correction terms due to the lens separation (S) and cornea thickness (tc). For the case of myopic correction, h=DWW/(nc−1)=0.333 D WW , where h is the ablation depth in microns and W is the ablation zone diameter in mm. For S=5.0 mm, f1=31.57 mm, f2=61.34 mm and tc=0.5 mm, the correction terms are about 8% and 2% respectively.

(b) The image shifted (db) about 0.45 mm for each of diopter change or each 14 microns change in h (for single-zone) and 11.1 microns (for 2-zone ablation), where we have used zone size W=6.5 mm. These results are derived from Eq. (5) and statement in (a).

(c) The diopter changes are quite sensitive to the changes of the indices of the cornea (nc), humor chamber (n1) and the lens (n). Given just 1% change of any of these indices, we estimate, based on Eq. (8), the diopter changes are −0.4 (due to cornea index change), +3.65 (due to index change in the lens) and −1.42 (due to index change in humor chambers). These changes are clinically important for the design and method to be proposed in the present patent. By the implications for above analysis, we are able to address the following as our examples of preferred embodiments.

### Preferred embodiment (A)

Correction of myopia may be done by decreasing the refractive index of the cornea such that the effective focal length, f1, increases and image position is shifted toward the retinal position, or b1 shifted to B=24.38 mm, b1−b>0, shown in Eq. (5). We note that this correction based on Eq. (8.c) for D3, also depends on the inverse difference of curvature radius of the cornea dr12=1/r1−1/r2. For larger dr12, this index effects will be larger. Eq. (8c) is based on dr12=0.02565 and r1=7.8 mm and r2=6.5 mm. For patients with smaller K-value (defined by 377/r1) of 40 the D1 value will increase from 0.4 (for K−43) per 1% change of nc to 0.55. And vice versa, D1 decreases to 0.0.32 for K=45. Therefore by measuring the K value, the r2 and the initial cornea index (nc), we should be able to predict the required index change of nc to achieve the diopter correction for a myopic or hyperopic patients.

### Preferred embodiment (B)

Similar effects as that of (B) may be achieved by changing the index of the humor chamber, either the anterior humor chamber (n1) or the posterior vitreous chamber (n2). Again, the amount of D4 depends also on the initial curvatures or radius of the lens (R1, R2) and the posterior radius of the cornea (r2), noting that D4 is almost independent on the anterior curvature of the cornea (r1), except a small amount due to a cross term of the overall effective focal length. Based on R1=10.2, R2=6.0, and r2=65. mm, we have derived Eq. (8.d) for D4. This effect increases for small R1 or R2, that is more effect may be achieved for young patients than old patients. For examples: for each 1% change of n1 or n2, we shall expect a change of D4 of −1.42 for age 60 and −1.63, −1.92, −2.29, −2.73 for younger ages of 50, 40, 30 and 20, respectively.

### Preferred embodiment (C)

The last item of Eq. (8) provides the effect of lens index change for the value of D5 in Eq. (8.e). For each 1% of change in lens index will achieve a correction depending on ages as follows: D5=(3.65, 3.9, 4.2, 4.6, 5.1) for age of (60, 50, 40, 30, 20). Eq. (8.e) is based on age at 60. It is important to note that when human gets old the lens index decreases due to the protein changes to more insolulable which has a lower index. In addition, the lens grows with larger R1 and R2 which also cause the patient to become hyperopia. The other age effect, presbyopia, is caused by the loss of elastic and space of the sclera tissue and ciliary body. By changing (increasing) the lens index, we shall be able to correct hyperopia and/or delaying the presbyopia to come. Reversely, we may decrease the index of the lens (n) for a young patient to correction the myopia.

Any or combination of the index changes in the cornea, humor chamber and lens shall allows us to correction patient's vision with a predictable results and re-treatment or enhancement is possible when patient's vision changed due to age. Enhancement of post-operative results by other methods such as PRK, LASIMK or LTK may be done by the index-changing method presented in the present patent.

### Preferred embodiment (D)

The means to change the refractive indices of the cornea, lens or humor chambers shall include, but not limited to: (a) using any eye drops or medications which may go into the chamber of the eye to modify the index but without causing other side effects, using injection of materials (liquid or gel form) which has a different index that that of the humor or lens of the eye; (b) using any optical, radiations or electromagnetic wave, high frequency ratio frequency wave, heating or cooling source to affect/change at least one of the indices of the eye; (c) using mechanical methods including the use of a device to physically change the pressure inside the eye such that its index changed by the circulating materials between the anterior and posterior chambers of the eye.; (d) using external implantation of materials with a predertermined index; (e) chemical methods in which the index of the eye may be changed by certain chemical reaction among the injected material and the materials in the eye. For example, the protein concentration or its property in the lens may be changed by any of the above methods. Similarly the optical property of the humor chamber may be changed by a medicine, or optical method. The implanted materials may be in any portion of the eye. Any methods may be used to modify the index of the eye either medical, mechanical, optical or chemical means should be within the scope of the present patent.

While the invention has been shown and described with reference to the preferred embodiments thereof, it will be understood by those skilled in the art that the foregoing and other changes and variations in form and detail may be made therein without departing from the spirit, scope and teaching of the invention. Accordingly, methods and apparatus, the ophthalmic applications herein disclosed are to be considered merely as illustrative and the invention is to be limited only as set forth in the claims.

I claim:

1. A method for performing vision correction comprising the steps of:
(a) measuring the refractive indices of the cornea, lens and humor chambers;
(b) measuring the patient's un-corrected vision and defining the required diopters;
(c) calculating the required index changes based on the measured data of initial index;
(d) selecting a means of changing the refractive index of at least one part of the eye consisting of the cornea, lens and humor chambers to treat vision problems of an eye.

2. A method for performing vision correction as claimed in claim 1, wherein the step of selecting said means of changing the refractive index includes medical, mechanical, optical or chemical method.

3. A method as claimed in claim 2, wherein said medical method includes the use of medicine which changes at least one of the refractive indices of the eye.

4. A method as claimed in claim 2, wherein said mechanical method includes the use of implantation of external material having a refractive index different from any of the index in cornea, lens or the humor chamber of the eye.

5. A method as claimed in claim 2, wherein said optical method includes the use of electromagnetic wave to change at least one of the refractive indices of the eye.

6. A method as claimed in claim 2, wherein said chemical method includes the use of chemical material to cause the change of the index inside the eye by chemical reaction among the injected materials and the material inside the eye.

7. A method for performing vision correction as claimed in claim 1, wherein said vision problems of an eye includes myopia, hyperopia, astigmatism and presbyopia.

8. An apparatus for performing vision correction comprising of:
(a) a device measuring the refractive indices of the cornea, lens and humor chambers;
(b) a device measuring the patient's un-corrected vision and defining the required diopters;
(c) a device providing a means of changing the refractive index of at least one part of the eye consisting of the cornea, lens and humor chambers to treat vision problems of an eye.

9. An apparatus for performing vision correction as claimed in claim 8, wherein said device providing means of changing the refractive index includes a medical, mechanical, optical and chemical device.
